(19) United States
(12) Patent Application Publication
(10) Pub. No.: US 2016/0068805 A1
(43) Pub. Date: Mar. 10, 2016

(54) METHOD TO EXPAND AND TRANSDUCE CULTURED HUMAN SMALL AND LARGE INTESTINAL STEM CELLS

(71) Applicants: THE REGENTS OF THE UNIVERSITY OF CALIFORNIA, Oakland, CA (US); THE UNITED STATES GOVERNMENT REPRESENTED BY THE DEPARTMENT OF VETERANS AFFAIRS, Washington, DC (US)

(72) Inventors: Martin G. Martin, Los Angeles, CA (US); James C.Y. Dunn, Santa Monica, CA (US); Matthias Stelzner, Pacific Palisades, CA (US)

(73) Assignees: THE REGENTS OF THE UNIVERSITY OF CALIFORNIA, Oakland, CA (US); THE UNITED STATES GOVERNMENT REPRESENTED BY THE DEPARTMENT OF VETERANS AFFAIRS, Washington, DC (US)

(21) Appl. No.: 14/853,564

(22) Filed: Sep. 14, 2015

Related U.S. Application Data
(63) Continuation of application No. PCT/US2014/030729, filed on Mar. 17, 2014.
(60) Provisional application No. 61/802,710, filed on Mar. 17, 2013.

(51) Int. Cl. C12N 5/071; A61K 35/38

(52) U.S. Cl. CPC C12N 5/068; A61K 35/38; C12N 2501/11; C12N 2501/40; C12N 2502/23; C12N 2501/415; A61K 48/00

(57) ABSTRACT

A cell culture media composition for support growth of human SI stem cells and epithelium without a feeder layer is presented. The media may also include growth factors including ENR and Y-27632 that support the survival of stem cell spheroid structures. The cell culture media compositions permit rapid growth of human small intestinal (SI) epithelium and stem cells, which leads to specific spheroid cell and enteroid morphology when grown in 3-D culture system.

### CROSS-REFERENCE TO RELATED APPLICATIONS

This application is a 35 U.S.C. § 111(a) continuation of PCT international application number PCT/US2014/030729 filed on Mar. 17, 2014, incorporated herein by reference in its entirety, which claims priority to, and the benefit of, U.S. provisional patent application Ser. No. 61/802,710 filed on Mar. 17, 2013, incorporated herein by reference in its entirety. Priority is claimed to each of the foregoing applications.

The above-referenced PCT international application was published as PCT International Publication No. WO 2014/153,294 on Sep. 25, 2014, which publication is incorporated herein by reference in its entirety.

### STATEMENT REGARDING FEDERALLY SPONSORED RESEARCH OR DEVELOPMENT

This invention was made with Government support under DK085535, awarded by the National Institutes of Health. The Government has certain rights in the invention. This work was supported by the U.S. Department of Veterans Affairs, and the Federal Government has certain rights in the invention.

### INCORPORATION-BY-REFERENCE OF COMPUTER PROGRAM APPENDIX

Not Applicable

### BACKGROUND OF THE INVENTION

1. Field of the Invention

This invention pertains generally to cell culturing media compositions and maintenance schemes, and more particularly to compositions and methods for maintaining stem cells and epithelial cells without a feeder layer and providing control over the expansion and morphology of stem cells and their descendant cells that make up the intestinal epithelium.

2. Description of Related Art

The intestinal epithelium is composed of a perpetually dividing epithelium composed of six primary cell types: the common absorptive enterocyte, the enteroendocrine cell, the mucous secreting goblet cell, the tuft cell, the M cell, and the Paneth cell. The intestinal crypt-niche interaction is thought to be essential to the function, maintenance, and proliferation of progenitor stem cells found at the bases of intestinal crypts. These stem cells are constantly renewing the intestinal epithelium by sending differentiated cells from the base of the crypts of Lieberkühn to the villus tips where they slough off into the intestinal lumen. The intestinal niche consists of various cell types, extracellular matrix, and growth factors and surrounds the intestinal stem and progenitor cells.

The molecular basis of intestinal stem cell (ISC) biology in both humans and mice is of great interest for potential clinical applications. During homeostasis, ISCs undergo cell division leading eventually to both a daughter progenitor cell that differentiates into the various absorptive and secretory cell lineages and a second ISC. However, ISCs are prone to injury by a range of stressful environmental insults, and the uninjured ISC populations expand by dividing symmetrically to produce two daughter ISCs to enable self-renewal. The extracellular signals that control the delicate balance between asymmetric and symmetric division are not yet well understood, particularly in humans.

The intimate contact between the small intestinal epithelium and the associated sub-epithelial myofibroblasts in both mouse and humans is generally thought to facilitate cross talk between the cell types and help to promote the growth and differentiation of the overlying epithelium. Mouse and human intestinal subepithelial myofibroblast (ISEMF) cells have been shown to support the growth of isolated intestinal epithelial cells.

Human epithelial cell cultures in conventional cell culture media used to grow murine intestinal epithelium, do not survive more than a few days. However, in the presence of mouse ISEMF cells, human epithelial cell cultures can be maintained for at least 60 days. By comparison, epithelial cultures lacking an ISEMF feeder layer die after only 2-3 days.

Previous attempts at culturing murine intestinal stem cells (ISCs) in a 3-D culture system were dependent on the addition of ENR (EGF, noggin, R-spondin1) factors. And these methods were not transferrable as it was observed that ENR factors failed to support the growth of human intestinal cells.

Other attempts at culturing human SI epithelial cells were dependent on a co-culture system requiring a murine and/or human intestinal subepithelial myofibroblast (ISMF) feeder layer, ENR and other factors (FGF10 and Wnt3a). The human intestinal subepithelial myofibroblasts are instrumental in orchestrating numerous functions in the intestine related to both health and disease.

Accordingly, a need exists for methods that allow human intestinal epithelial cells and intestinal stem cells to be cultured reliably. There is also a need for methods to rapidly expand the number of human small and large bowel mucosa for either research, therapeutic or toxicological use. There is also a need for methods that allow for the transduction of the human SI/LI ISC populations with lentiviral and potentially other viruses. The present invention satisfies these needs as well as others and is an improvement in the art.

### BRIEF SUMMARY OF THE INVENTION

The present invention generally provides methods for producing cell culture media compositions that will allow the propagation of intestinal stem cells by symmetric division as well as support for growth and division of other normal and abnormal cells such as epithelial cells.

While currently available culture methods for human SI/LI epithelial ISCs are suited to maintain the cells in cell culture for several weeks, the present invention allows for the rapid expansion of stem cell numbers as well as to allow transduction with viral agents like lentivirus. It is therefore possible to produce genetically modified SI/LI epithelial ISCs that can be grown in culture and amplified. This is a pivotal step for the clinical application of these stem cells. With the ability to support growth and cell propagation, it will be possible to insert transgenes into human small or large intestinal mucosal stem cells as a method for gene therapy. These cells can then be propagated in vitro in preparation for implantation in human patients. For example, this methodology could permit the harvesting of human cells to modify them genetically and then re-implant them into the same patient in an effort to cure a genetically-based gastrointestinal disease condition such as a diarrhea, inflammatory bowel disease, cancer or other disorders due to a genetic defect. The transduced stem cells may be introduced or integrated into the native mucosa by injection of cells or by surface implantation into an area of the intestine that had its resident mucosal cells removed by a biochemical or physical intervention (e.g., chemical debridement, radiation, mechanical debridement, etc.).

Moreover, the development of this human SI/LI in vitro model will provide human models for research and toxicology assessment of drugs and pathways.

The present methodology and culture media will permit health practitioners to harvest human SI/LI epithelia ISCs and culture them to quickly expand cell numbers for research, therapeutic and toxicology purposes. It will also be possible to modify the cells genetically before use, for example before implantation. Moreover, this media can also be used separately from its transduction inducing capabilities, and be used to massively expand SI/LI ISCs in a very rapid manner.

According to one aspect of the invention, a culture media for epithelial cells is provided by incubating human or murine intestinal subepithelial myofibroblast (ISEMF) cells in normal low glucose media with 10% FBS, insulin, transferrin, and EGF for a period of 4-7 days. The incubation condition for optimal growth includes 10% CO₂, and not the usual 5% that is generally used in most culture conditions. Thereafter, incubation media is collected and any residual ISEMF cells are removed by filtering the media through a filter. In one embodiment, Y-27632, and ENR factors are added to the incubation media (MF-CM).

According to another aspect of the invention, the use of specific MF-CM can be used to support the growth of human SI/LI epithelium by promoting the symmetric division of ISCs leading to the expansion of spheroid structures, while the removal of MF-CM and the addition of other factors induces development of more differentiated SI/LI cells with these enteroid structures.

Another aspect of the invention is to provide a cell culture media formulation that will maintain growth of spheroids and that can be used to convert spheroids to enteroids.

A further aspect of the invention is to provide a media that will support massive expansion of intestinal epithelium isolated by endoscopic biopsy as well as allow growth of pre-malignant and malignant epithelial cells obtained by biopsy/resection for assessment to responsiveness to anti-neoplastic reagents.

Another aspect of the invention is to provide a cell culture media and methods that can be used to grow intestinal epithelial cells obtained by biopsy/resection from patients with inflammatory bowel disease for assessment to responsiveness to anti-inflammatory reagents, or reagents that alter epithelial function, including tight junction formation.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a schematic flow diagram of one process for producing intestinal cell culture media (MF-CM) according to one embodiment of the invention.

### DETAILED DESCRIPTION OF THE INVENTION

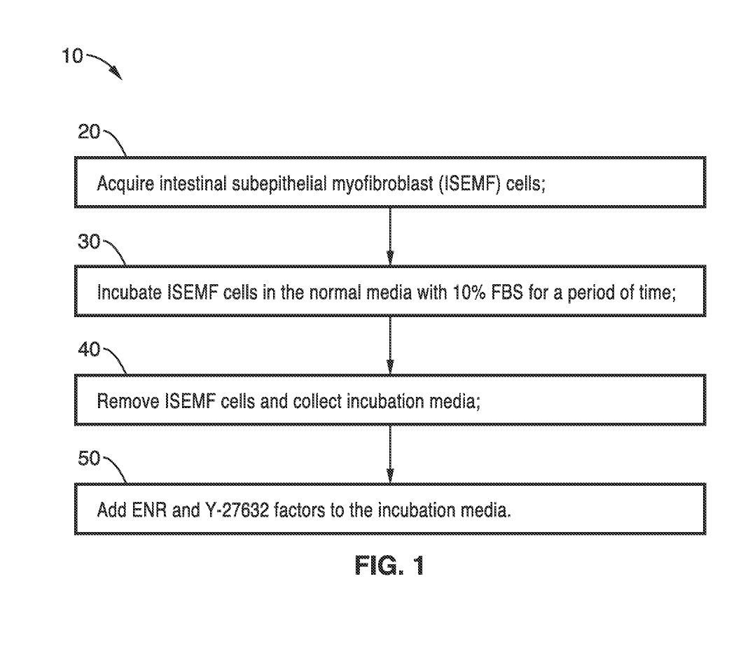

Referring more specifically to the drawings, for illustrative purposes an embodiment of the method for producing ISEMF conditioned culture media compositions of the present invention that will support human SI/LI epithelial cells and human ISCs is described and depicted generally in FIG. 1. It will be appreciated that the methods may vary as to the specific steps and sequence and the media compositions may vary as to elements without departing from the basic concepts as disclosed herein. The method steps are merely exemplary of the order in which these steps may occur. The steps may occur in any order that is desired, such that it still performs the goals of the claimed invention.

Turning now to FIG. 1, a flow diagram of one embodiment of method 10 for generating a specific cell culture media is generally shown. The MF-CM cell culture media that is produced by the methods has properties that allow human small and large intestinal (SI/LI) mucosal stem cells to grow rapidly, which leads to specific spheroid cell morphology and characteristics when grown in 3-D culture system. The media of the present invention can grow human SI/LI stem cells and epithelium without a feeder layer.

This is performed using condition media (CM) developed from incubating normal media (with 10%FBS) on human intestinal subepithelial myofibroblast lines. Conventional media is bathed on the ISEMF cells for several days (e.g. 4-7 days), and this conditioned media is called MF-CM. If this culture media is applied to human SI epithelium, growth and survival of these cells is observed for a significant period of time (e.g. 1-2 months). Furthermore, with the addition of two other factors (Y-27632, and ENR) it has been shown that the SI/LI epithelium is primarily comprised of spheroid structures that can be rapidly grown and expand by splitting these structures. The factors (Y-27632, ENR) enhance the growth of these spheroid structures, and the R-Spondin component is certainly necessary.

At block 20 of FIG. 1, one or more intestinal subepithelial myofibroblast lines are acquired. Although human ISEMF cells are preferred, other suitable sources of ISEMF cells may be used. As used herein, “source” means any living organism to which ISEMF cells may be acquired. Other sources may include, but are not limited to, monkeys, cows, goats, sheep, mice, rats, cats, dogs, horses, hamsters, and any transgenic animals.

The ISEMF cells are typically acquired by isolating the cells from the intestines of a source. As used herein “isolated” defines a substance, for example an intestinal stem cell population or epithelial cell population, that is separated from contaminants (for example, contaminants are substances or cells that are no intestinal stem cells).

The acquired ISEMF cells are placed into conventional culture media containers and preferably incubated with normal low glucose cell culture media. In one embodiment, the normal culture medium includes approximately 10% Fetal Bovine Serum (FBS).

Cell culture media provides the nutrients necessary to maintain and grow cells in a controlled, artificial and in vitro environment. However, cells in different tissues of the mammalian body often require the use of different media formulations that may vary depending on the particular cellular requirements of the cells to be supported by the media. Cell culture media formulations have been well documented in the literature and a number of media products are commercially available.

The preferred normal cell culture media that is selected at block 20 is one that is particularly suitable for suspension culture of intestinal epithelial cells and intestinal stem cells. A typical culture medium is composed of a complement of amino acids, vitamins, inorganic salts, glucose, and serum as a source of growth factors, hormones, and attachment factors. In addition to nutrients, the medium also helps maintain pH and osmolality.

The normal cell culture media formulations may be supplemented with a range of additives, including undefined components such as fetal bovine serum (FBS) (10-20% v/v) or extracts from animal embryos, organs or glands (0.5-10% v/v). While FBS is one of the most commonly used supplements in animal cell culture media, other serum sources are also routinely used, including those from newborn calf, horse and human sources.

In one embodiment, the preferred media for the development of human intestinal subepithelial myofibroblast cells is a low glucose media with 10% FBS and with EGF, transferrin, and insulin as additives.

The ISEMF cells are incubated in the normal low glucose culture medium for a period of time at block 30 of FIG. 1. The preferred incubation period is approximately 4 days to 7 days. For optimal potency, the condition media should be grown in an incubator at 10% CO₂.

At block 40, the ISEMF cells are removed from the incubation media and the incubation media is collected. The incubation media is preferably filtered to remove any contaminants. The collected media can be used alone or may have other known growth promoting factors and nutrients added so that the media can be tailored to the type of target cells that will be supported by the conditioned media.

In the embodiment of FIG. 1, at block 50 growth factors (Y-27632, ENR) are optionally added to the incubation media for use with stem cells. As used herein, a “stem cell” defines a less differentiated stem cell that can give rise to distinct (genotypically and/or phenotypically) further differentiated progeny cells.

It has been demonstrated that MF-CM is primarily responsible for the promotion of these spheroid structures, which are enriched in stem cells and devoid of progenitor and differentiated cells of the SI/LI like enterocytes, Paneth cells, goblet cells and enteroendocrine cells. If MF-CM is removed from the 2 and 3-D or 2-D culture and CHIR99021/Y-27632 and other factors (ENR, Wnt3a and FNF10) are retained, the spheroid structures develop into enteroid structures that are composed of stem/progenitor and differentiated cells of the SI/LI. We have also found that these spheroid structures that are enhanced by MF-CM can be transduced by lentiviruses, therefore allowing genes within these cells to be manipulated.

It has been shown that subepithelial myofibroblast (ISEMF) conditioned media (MF-CM) is required for optimal long-term growth and passage of intestinal epithelial stem cell populations as spheroid structures. Spheroids or spheroid structures refer to balloon shaped structures composed of intestinal epithelial stem cells. It has also been observed that the prostaglandin (PGE2) is produced by ISEMF and is present in the MF-CM and is required for the induction of spheroids. The PGE2 works synergistically with a growth factor named R-Spondin (RSPO) that is also secreted by ISEMF cells to the media. However, while PGE2 and RSPO are required for spheroid formation, they have limited long-term growth and ability to passage. The spheroids (or stem cells that make up the spheroids) can be induced to differentiate into normal intestinal epithelium by removing MF-CM and applying the growth factor Wnt3a and or the optional addition of CHIR99021, ENR and FHG10. The intracellular signals that are induced by factors present in the MF-CM are mediated by two pathways (Ras/Raf/MEK/ERK1/2 and BMK/MEK/ERK5 axis).

It has been demonstrated that R-Spondin, PGE2 and other factor(s) secreted by ISMEF induce symmetric cells division and lineage expansion of stem cells and that these mediators have a role in the pathogenesis of chronic enteric infection, malignancy, inflammation and radiation-induced damage, and that spheroids are in vitro depictions of what is likely occurring in vivo.

For example, it is shown that the use of ISEMF-conditioned media is important for in vitro growth of intestinal epithelium isolated from a patient with familial adenomatous polyposis (FAP). FAP is a common monogenic disorder that results in colon and small bowel cancer and is generally secondary to autosomal dominant loss of function of mutations in genes in the APC pathway, genes that are commonly mutated in patients with common non-inherited forms of colon cancer.

All usual methods to sustain long-term growth of the small bowel epithelium in the enteroid and spheroid state from a patient with FAP were found to be ineffective (including R-spondin and PGE2), however, the epithelium can be massively expanded with MF-CM that induces spheroid formation. It has been demonstrated that once the epithelium is expanded in a spheroid state, they can be converted back into enteroids for more functional assessment.

The invention may be better understood with reference to the accompanying examples, which are intended for purposes of illustration only and should not be construed as in any sense limiting the scope of the present invention as defined in the claims appended hereto.

### Example 1

In order to demonstrate the operational principles of the culture media and methods, ISEMF cells were isolated from five-day old wild type C57BL/6 neonates, in which mesenchyme-rich small intestinal organoids were harvested using gentle enzymatic digestion and plated at a density of 5,000 per mL of ISEMF media. This media consisted of Dulbecco's modified Eagle medium (DMEM)/Low Glucose/GlutaMAX (Invitrogen, Carlsbad, Calif.) with 10% FBS (Invitrogen), lx Antibiotic-Antimycotic (Invitrogen), 0.25 U/mL insulin (Sigma, St. Louis, Mo.), 10 mg/mL transferrin (Sigma), and 20 ng/mL recombinant murine epidermal growth factor (EGF, Peprotech, Rocky Hill, N.J.). After ISEMF cells attached and formed colonies, they were subsequently passaged and expanded using standard cell culture techniques. In preparation for co-culture, ISEMF cells were seeded into 48-well cell culture plates, with and without pre-treatment with gelatin, and allowed to grow to confluency. For conditioned media (CM) experiments, ISEMF media was replaced after the cells formed a confluent monolayer. The media was collected after 5 days of incubation, passed through a 0.2 mm pore size filter (Pall Corporation, Port Washington, N.Y.), and used as ISEMF CM.

The identity and purity of isolated ISEMF cells was determined by quantitative real-time polymerase chain reaction (qPCR) and immunofluorescence. Neonatal murine cells were employed in the majority of experiments. Adult murine ISEMF cells were identically isolated and grown from 2-month-old C57BL/6 wild-type mice in order to compare ISEMF populations.

### Example 2

To further demonstrate the supportive effects of ISEMFcells in intestinal epithelial culture, small and large intestinal crypts were cultured in the presence and absence of ISEMF cells. When treated with media lacking exogenous Rspo1, crypt monocultures did not form enteroids and died within two days of plating, highlighting the necessity for Rspo1. However, it was observed that crypts co-cultured with ISEMF cells successfully formed enteroids even in the absence of exogenous Rspo1.

Crypts were grown with and without ISEMF-conditioned culture medium ISEMF-CM to assess the necessity of interaction between ISEMF cells and the epithelial cells. ISEMF CM was collected from confluent ISEMF cells in monoculture after 5 to 7 days of incubation as described earlier and filtered to eliminate cell contamination. Crypts grown with media containing ISEMF-CM were 3 times larger (0.08760.029 mm² versus 0.03860.016 mm²; p=0.03) than those without conditioned media. These data suggest that the ISEMF cells are actively secreting soluble factors that enhance ISC growth. In another illustration, ISEMF-conditioned Complete Crypt Medium (IC-CCM) was prepared in the following manner: ISEMF cells were sub-cultured into a T-75 culture flask (Corning, Tewksbury, Mass.) and treated with ISEMF medium. The media was removed after one week of incubation and stored in a 4° C. refrigerator for subsequent use. ICCCM was constituted prior to each use by preparing a 1:1 mixture of the spent ISEMF medium with doubly-concentrated CCM, such that the final concentration of the specific nutrients and growth factors above was unchanged. 10 μM Y-27632 (Sigma) and ENR factors were also added.

Small intestinal crypts were isolated and suspended as above. They were then plated into 48-well Nunclon Delta-treated cell culture plates (Thermo Scientific, Waltham, Mass.) and treated with IC CCM. The resultant epithelial units were sub-cultured every 4-5 days with a 1:2 split ratio, using type XI-S collagenase (Sigma) and gentle mechanical disruption. They were then resuspended in collagen gel and re-plated followed by continued IC-CCM treatment.

From the discussion above it will be appreciated that the invention can be embodied in various ways, including but not limited to the following:

1. A cell culture medium composition for epithelial and intestinal stem cells, comprising normal cell culture media incubated with human intestinal subepithelial myofibroblast cells for a period of time.

2. A composition as recited in any previous embodiment, further comprising an additive of at least one intestinal cell growth factor.

3. A composition as recited in any previous embodiment, wherein the additive is selected from the group of additives consisting of epidermal growth factor (EGF), noggin, R-spondin1 and combinations thereof.

4. A composition as recited in any previous embodiment, wherein the additive is selected from the group of additives consisting of Y-27632, a combination of ENR and Y-27632, CHIR9902, a combination of CHIR99021 and Y-27632, and GSKi.

5. A method of growing human small and large intestinal (SI/LI) stem cells or epithelium without a feeder layer, comprising: (a) incubating intestinal subepithelial myofibroblast (ISEMF) cells in normal cell culture media with FBS for period of four days to seven days; (b) removing the ISEMF cells from the incubated cell culture media wherein conditioned media (MF-CM) is produced; and (c) growing human SI or LI crypts, stem cells or epithelium in the conditioned media.

6. A method as recited in any previous embodiment, further comprising: promoting the symmetric division of intestinal stem cells (ISCs) leading to the expansion of spheroid structures the MF-CM to support growth; and replacing the MF-CM media with normal cell culture media to induce more differentiated SI/LI cells with enteroid structures.

7. A method as recited in any previous embodiment, further comprising: adding ENR and Y-27632 to the MF-CM; wherein the SI/LI epithelium is primarily spheroid structures that can be rapidly grown and expanded by splitting these structures; and wherein the ENR and Y-27632 support survival of the spheroid structures.

8. A method as recited in any previous embodiment, further comprising: adding a growth factor to the MF-CM selected from the group of factors consisting of epidermal growth factor (EGF), noggin, R-spondin1 and combinations thereof.

9. A method as recited in any previous embodiment, wherein spheroid structures produced by cells in MF-CM are enriched in stem/progenitor cells and devoid of differentiated cells of the small and large intestine.

10. A method as recited in any previous embodiment, wherein if the MF-CM is removed from the 2-D or 3-D culture and ENR, CHIR99021, Wnt3a, Y-27632 and FHG10 factors are retained, the spheroid structures develop into enteroid structures that are composed of stem/progenitor and differentiated cells of the intestinal epithelium.

11. A method of as recited in any previous embodiment, wherein due to enhancement by MF-CM, the spheroid structures can be transduced by lentiviruses, thereby allowing manipulation of genes within these cells.

12. A method of producing and growing genetically modified small/large intestine (SI/LI) epithelial intestine stem cells (ISCs) without a feeder layer, comprising: (a) incubating intestinal subepithelial myofibroblast (ISEMF) cells in normal cell culture media with about 10% FBS, low glucose, insulin, transferrin and EGF for about four to seven days; (b) removing the ISEMF cells from the incubated cell culture media wherein conditioned media (MF-CM) is produced; (c) growing SI/LI crypts, stem cells or epithelium in the conditioned media; (d) adding at least one growth initiator to the MF-CM media; and (e) inserting human or non-human transgenes into human SI/LI stem cells as a method for gene therapy.

13. A method as recited in any previous embodiment, further comprising: propagating the genetically modified cells in vitro in preparation for implantation into human patients.

14. A method as recited in any previous embodiment, further comprising: amplifying the genetically modified cells: harvesting the genetically modified cells; and implanting the modified cells into the same patient in an effort to cure a genetically-based disease condition caused by a genetic defect.

15. A method as recited in any previous embodiment, further comprising: integrating the transduced stem cells into the native mucosa by injection of cells or by surface implantation into an area of the intestine that had its resident mucosal cells removed.

16. A method as recited in any pervious embodiment, wherein the growth initiator is selected from the group of initiators consisting of Y-27632, a combination of ENR and Y-27632, CHIR9902, a combination of CHIR99021 and Y-27632, and GSKi.

17. A method as recited in any previous embodiment, wherein the growth initiator is selected from the group of initiators consisting of epidermal growth factor (EGF), noggin, R-spondin1 and combinations thereof.

18. A composition as recited in any previous embodiment, further comprising an additive of at least three intestinal cell growth factors, and an anti-apoptotic reagent.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A cell culture medium composition for epithelial and intestinal stem cells, comprising normal cell culture media incubated with human intestinal subepithelial myofibroblast cells for a period of time.

2. A composition as recited in claim 1, further comprising an additive of at least one intestinal cell growth factor.

3. A composition as recited in claim 2, wherein said additive is selected from the group of additives consisting of epidermal growth factor (EGF), noggin, R-Spondin1 (ENR) and combinations thereof.

4. A composition as recited in claim 2, wherein said additive is selected from the group of additives consisting of Y-27632 and a combination of ENR and Y-27632.

5. A method of growing human small and large intestinal (SI/LI) stem cells or epithelium without a feeder layer, comprising:
(a) incubating intestinal subepithelial myofibroblast (ISEMF) cells in normal cell culture media with FBS for period of four days to seven days;
(b) removing the ISEMF cells from the incubated cell culture media wherein conditioned media (MF-CM) is produced; and
(c) growing human SI or LI crypts, stem cells or epithelium in the conditioned media.

6. A method as recited in claim 5, further comprising:
promoting the symmetric division of intestinal stem cells (ISCs) leading to the expansion of spheroid structures with the MF-CM to support growth; and
replacing the MF-CM media with normal cell culture media to induce more differentiated SI/LI cells with enteroid structures.

7. A method as recited in claim 5, further comprising:
adding ENR and Y-27632 to the MF-CM;
wherein the SI/LI epithelium is primarily spheroid structures that can be rapidly grown and expanded by splitting; and
wherein the ENR and Y-27632 support survival of the spheroid structures.

8. A method as recited in claim 5, further comprising:
adding a growth factor to the MF-CM selected from the group of factors consisting of epidermal growth factor (EGF), noggin, R-Spondin1 and combinations thereof.

9. A method as recited in claim 8, wherein spheroid structures produced by cells in MF-CM are enriched in stem/progenitor cells and devoid of differentiated cells of the small and large intestine.

10. A method as recited in claim 8, wherein if the MF-CM is removed from the 2 or 3-D culture and ENR, CHIR99021, Wnt3a, Y-27632 and FHG10 factors are retained, the spheroid structures develop into enteroid structures that are composed of stem/progenitor and differentiated cells of the small and large intestinal epithelium.

11. A method as recited in claim 8, wherein due to enhancement by MF-CM, the spheroid structures can be transduced by lentiviruses, thereby allowing manipulation of genes within these cells.

12. A method of producing and growing genetically modified small/large intestine (SI/LI) epithelial intestine stem cells (ISCs) without a feeder layer, comprising:
(a) incubating intestinal subepithelial myofibroblast (ISEMF) cells in normal cell culture media with about 10% FBS, low glucose, insulin, transferrin and EGF for about four to seven days;
(b) removing the ISEMF cells from the incubated cell culture media wherein conditioned media (MF-CM) is produced;
(c) growing SI/LI crypts, stem cells or epithelium in the conditioned media;
(d) adding at least one growth initiator to the MF-CM media; and
(e) inserting human or non-human transgenes into human SI/LI stem cells as a method for gene therapy.

13. A method as recited in claim 12, further comprising:
propagating the genetically modified cells in vitro in preparation for implantation into human patients.

14. A method as recited in claim 12, further comprising:
amplifying the genetically modified cells;
harvesting the genetically modified cells; and
implanting the modified cells into the same patient in an effort to cure a genetically-based disease condition caused by a genetic defect.

15. A method as recited in claim 14, further comprising:
integrating the transduced stem cells into the native mucosa by injection of cells or by surface implantation into an area of the intestine that had its resident mucosal cells removed.

16. A method as recited in claim 12, wherein said growth initiator is selected from the group of initiators consisting of ENR, Y-27632 and a combination of ENR and Y-27632.

17. A method as recited in claim 12, wherein said growth initiator is selected from the group of initiators consisting of epidermal growth factor (EGF), noggin, R-spondin1 and combinations thereof.
